(19) United States
(12) Patent Application Publication
(10) Pub. No.: US 2026/0094724 A1
(43) Pub. Date: Apr. 2, 2026

(54) SYSTEM AND METHOD FOR DISEASE MODELING

(71) Applicant: STCHealth, LLC, Phoenix, AZ (US)

(72) Inventors: Samuel McGee, Phoenix, AZ (US); Zoey Gabbard, Phoenix, AZ (US)

(73) Assignee: STCHealth, LLC, Phoenix, AZ (US)

(21) Appl. No.: 19/343,831

(22) Filed: Sep. 29, 2025

Related U.S. Application Data
(60) Provisional application No. 63/700,944, filed on Sep. 30, 2024.

(51) Int. Cl. G16H 50/50

(52) U.S. Cl. CPC G16H 50/50

(57) ABSTRACT

Systems and methods including one or more processors and one or more non-transitory storage devices storing computing instructions configured to run on the one or more processors and perform acts of defining at least one of one or more classes, one or more objects, or one or more functions; generating an environment for a for-loop simulation; generating one or more agents; running the for-loop simulation using the at least one of the one or more classes, the one or more objects, or the one or more functions; the environment; and the one or more agents; and generating a visualization of the for-loop simulation. Other embodiments are disclosed herein.

### CROSS REFERENCE TO RELATED APPLICATIONS

This application claims priority to U.S. Provisional Application No. 63/700,944, filed Sep. 30, 2024 and entitled SYSTEM AND METHOD FOR DISEASE MODELING, which is herein incorporated by this reference in its entirety.

### TECHNICAL FIELD

This disclosure relates generally to computer modeling of disease activity and more specifically pertains to modeling new diseases and the effects of interventions on disease activity.

### BACKGROUND

In the past, modeling disease activity within a population can include factors such as disease transmission and mortality, but this approach does not consider the complexities of the modern world. For example, previous models do not account for properties of new diseases. As another example, previous models are incapable of considering the effect of interventions (e.g., medications and/or medical devices) on transmission of the disease. However, as disease processes are complex, additional variables can be incorporated to increase a fidelity and/or accuracy of such models by incorporating additional variables that capture new attributes of the agents or their environment.

Adding new variables to a disease model can present several technical challenges. First, incorporating new parameters increases a dimensionality of a model, which in turn expands computational burdens on processors. Past simulations with higher dimensionality can require more processing time, more memory allocation, and greater storage capacity, which can strain available computational resources and limit the feasibility of large-scale geographic studies.

Second, an addition of new variables often necessitates restructuring of existing model architectures to accommodate new data. Legacy code bases may not be readily adaptable to the inclusion of new data types, classes, or attributes, thereby leading to significant overhead in terms of code modification, debugging, and model validation. This structural complexity can hinder the scalability and reusability of models across different studies. Further, legacy variable can be calculated using clunky methods that put undue burdens on processors and suck up compute needed for new variables.

Third, a presence of additional variables can lead to challenges in data management and interoperability. New variables can originate from heterogeneous data sources, which can vary in terms of resolution, quality, and format. Aligning this new data with a structure of an existing model can require substantial preprocessing and transformation.

In view of the above, there is a need for a new system and method for modeling the activity of a disease within a population.

### SUMMARY

A number of embodiments can include a system. The system can include one or more processors and one or more non-transitory computer-readable storage devices. The one or more non-transitory computer-readable storage devices can store computing instructions. The computing instructions can be configured to communicate with the one or more processors and cause the one or more processors to perform defining at least one of one or more classes, one or more objects, or one or more functions; generating an environment for a for-loop simulation; generating one or more agents; running the for-loop simulation using the at least one of the one or more classes, the one or more objects, or the one or more functions; the environment; and the one or more agents; and generating a visualization of the for-loop simulation.

Various embodiments include a method. The method can be implemented via execution of computing instructions configured to run at one or more processors and/or configured to be stored at non-transitory computer-readable media The method can comprise defining at least one of one or more classes, one or more objects, or one or more functions; generating an environment for a for-loop simulation; generating one or more agents; running the for-loop simulation using the at least one of the one or more classes, the one or more objects, or the one or more functions; the environment; and the one or more agents; and generating a visualization of the for-loop simulation.

Various embodiments can include an article of manufacture. The article of manufacture can include a non-transitory, tangible computer readable storage medium. The non-transitory, tangible computer readable storage medium can store instructions that, in response to execution by a computer, cause the computer to perform operations comprising defining at least one of one or more classes, one or more objects, or one or more functions; generating an environment for a for-loop simulation; generating one or more agents; running the for-loop simulation using the at least one of the one or more classes, the one or more objects, or the one or more functions; the environment; and the one or more agents; and generating a visualization of the for-loop simulation.

### BRIEF DESCRIPTION OF THE DRAWINGS

To facilitate further description of the embodiments, the following drawings are provided in which:

FIG. 1 illustrates a flowchart for a method, according to an embodiment;

FIG. 2 illustrates a representative block diagram of a system, according to an embodiment; and

FIG. 3 illustrates a representative block diagram of a system, according to an embodiment.

### DESCRIPTION OF EXAMPLES OF EMBODIMENTS

Agent-based modeling (“ABM”) is a computational approach that can be used to simulate interactions of individual agents (e.g., humans, animals, or pathogens) within a defined environment. In epidemiology, ABM can be used for studying a spread of diseases, how interventions can influence outcomes, and how heterogeneous behaviors among and/or between agents can affect disease trajectories. ABM can allow researchers to model behavior of individual hosts through their movements, interactions, and transmission of disease within a population. Unlike traditional epidemiological models (e.g., compartmental models), ABMs can incorporate heterogeneity among agents and account for complex dynamics among and/or between agents.

In many embodiments, the techniques described herein can provide a practical application and several technological improvements. In some embodiments, the techniques described herein can provide for a disease model that takes into account properties of new diseases and/or interventions. These techniques can provide for a significant improvement over conventional approaches of disease modeling, such as only modeling properties of older diseases (e.g., virulence, mortality, etc.). Moreover, these estimates are technical improvements over other possible approaches because a more accurate model of disease activity is created. In this way, the techniques described herein can avoid problems with inaccurate and outdated models.

In various embodiments, techniques described herein cannot be practically performed in the mind of a human. A disease model can comprise thousands or even millions of agents, each of which can have multiple attributes (e.g., age, health status, location, and/or susceptibility to infection). Tracking the state of each agent, updating interactions between agents at every time step, and computing probabilistic outcomes involve extensive calculations across a high-dimensional parameter space. The volume of calculations grows exponentially as new variables are introduced or as the number of simulated agents increases.

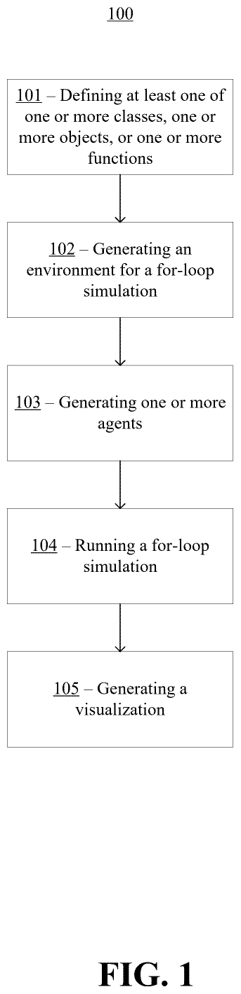

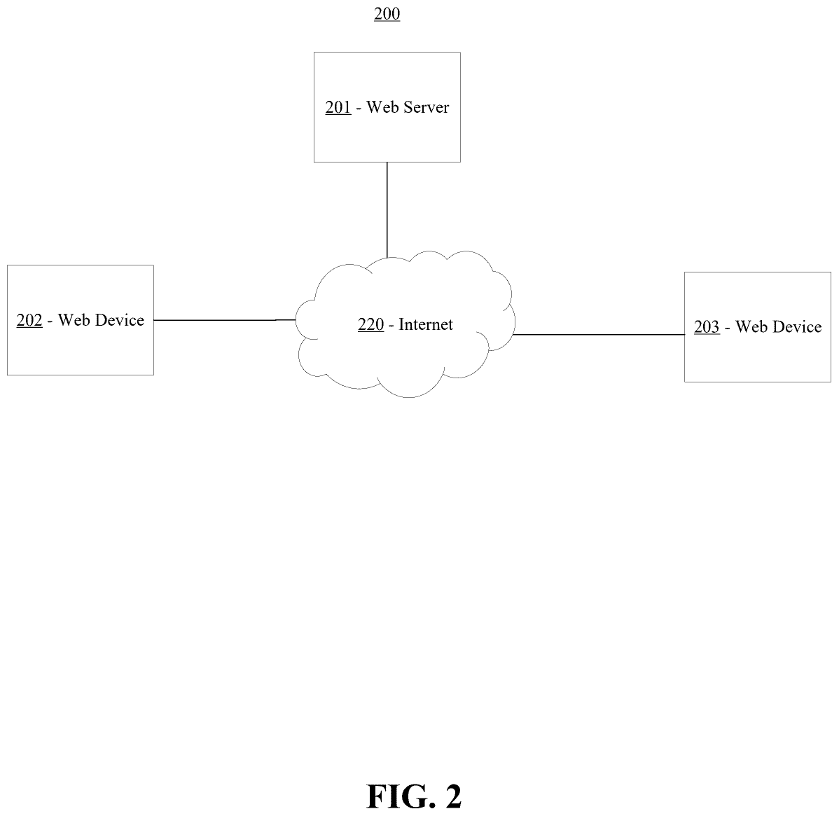

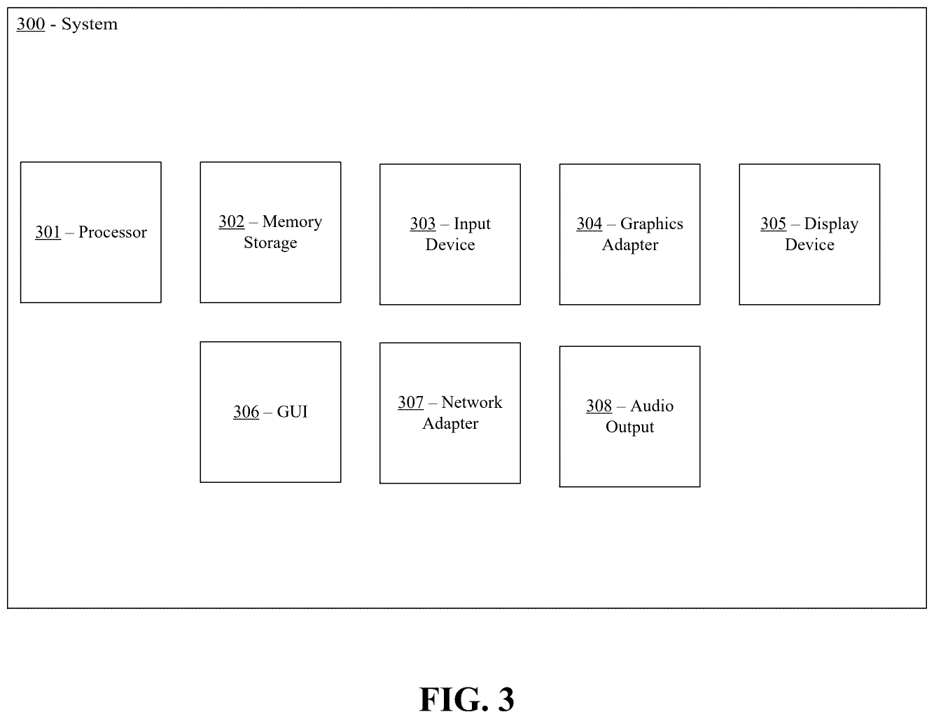

Turning ahead in the drawings, FIG. 1 illustrates a flow chart for a method 100, according to an embodiment. Method 100 is merely exemplary and is not limited to the embodiments presented herein. Method 100 can be employed in many different embodiments or examples not specifically depicted or described herein. In some embodiments, the activities of method 100 can be performed in the order presented. In other embodiments, the activities of method 100 can be performed in any suitable order. In still other embodiments, one or more of the activities of method 100 can be combined or skipped. In many embodiments, system 300 (FIG. 3) can be suitable to perform method 100 and/or one or more of the activities of method 100. In these or other embodiments, one or more of the activities of method 100 can be implemented as one or more computer instructions configured to run at one or more processing modules and configured to be stored at one or more non-transitory memory storage modules. Such non-transitory memory storage modules can be part of a computer system such as web server 201 (FIG. 2) and/or web devices 202, 203. The processing module(s) can be similar or identical to the processing module(s) described above with respect to computer system 300 (FIG. 3).

In many embodiments, method 100 can comprise an activity 101 of defining at least one of one or more classes, one or more objects, or one or more functions. In ABMs, classes can be used to represent different components of the model, such as agents, environments, and/or one or more processes that govern interactions between agents. These classes provide a structure for defining behavior of agents and managing a simulation's complexity. In ABMs, agents can be used to represent people, animals, or other entities. Each agent can have a unique set of characteristics (e.g., age, health status, social behavior, etc.) that is defined in one or more classes. These agents interact with each other and their environment, which helps in simulating real-world dynamics more accurately. In many embodiments, an age-dependent mortality class can be used to model natural mortality in a population. For example, the age-dependent mortality class can eliminate a predetermined percentage of a population of agents each time step. In many embodiments, elimination can result in an agent being removed from a population dataframe. In ABMs, functions are used to define behaviors and attributes of classes. Functions can be seen as forming the logic behind how agents, environments, and simulations operate. Functions can be encapsulated within classes, and can allow for agent movement, agent interactions, disease transmission, and/or environment updates. An age-dependent mortality class can have a function comprising a number of inputs: a self-object, a float between zero and one that assigns a probability of agent death, a Boolean value that determines whether or not to skew elimination by age, a float value representing a probability that an agent over an age of sixty years is eliminated, a 1D array of Boolean values that add additional conditions for selecting agents, a Boolean value that determines whether or not to return an array that stores information on each agent that is eliminated, and/or an integer value that sets a seed for the simulation. A function for an age-dependent mortality class can operate to assign a probability for mortality that is weighted by agent age.

An age-dependent mortality class can separate an agent population into agents over sixty years and agents under sixty years. The age-dependent mortality class can assign each agent a value between zero and one and can check to see when the value is above a probability of elimination for that age cohort. The check can begin by assigning a Boolean value to the agent. A mask array of zeros can then be applied to a population of agents, and a conditional Boolean array can be compared against the Boolean value assigned to the agent to determine that agent's viability of selection for elimination. If a Boolean value is false, then a random probability between zero and one is given to an agent and compared to a base proportion of elimination. Agents that have proportions below the base proportion are then eliminated. An array storing information of agents that are removed can be returned to track demographics and totals for a simulation. In many embodiments, operating time for an age-dependent mortality class can be lessened by using Boolean array masks as a way of classification instead of if-loops. This substitution can reduce burdens on a processor, thereby leading to a faster model and/or a model that allows more processing power to be allocated to new variables.

In many embodiments, a multi-inheritance class can be used in an ABM to represents a monogamous mammal capable of movement (e.g., a human). The multi-inheritance class can define a population wide mortality rate supplied by a user of a system. A population wide mortality rate can be divided into two or more buckets based on age. For example, a population wide mortality rate can stratify a mortality rate into agents under 60 years and 60 years and above. This can allow agent generation and for-loop processing to be sped up because calculating and processing of individual mortality rates for each agent (a data and processor intensive process) can be simplified into an age check for the agent. This, in turn, can allow for incorporation of age-related variables while maintaining or improving model compute burdens on a processor.

In many embodiments, a transition class can allow for transitions between disease states to occur. For example, disease states can comprise contagious, infected, susceptible, immune, dead, and/or quarantined. In many embodiments, immunity can be given to agents that survive a disease cycle. Diseases stages can be tracked for reporting purposes. For example, disease states can be tracked through Boolean and/or integer columns specified in a Python object (e.g., a dataframe). Each disease stage (including immunity) can last for a user-specified length of time. For example, a contagious stage can last two time steps while immunity can last ten time steps. Disease stages can be tracked using a constructor function. For example, a constructor function can initialize the following attributes to an agent population: a counter for tracking an infection stage, a counter for tracking a contagious stage, a counter for tracking an immunity stage, and/or a counter for tracking quarantine stage. The AMB can call a ticker method decrement a counter of disease stage.

Agents can be transitioned between an initial state and a target state. In many embodiments, an initial state and a target state can be represented by an integer array. The integer array can represent one or more of six parameters: susceptible, infected, contagious, quarantined, dead, and/or immune. A susceptible agent can comprise an agent that can be infected, but not currently infected. An infected agent can comprise an agent that is incubating a disease, but not contagious. An infected agent tests positive for the disease. A contagious agent can comprise an agent that is able to spread a disease to other agents through contact on a vertex. A quarantined agent can comprise an agent that has tested positive for a disease but not spreading a disease. A quarantined agent does not move vertices after a time step. A dead agent can comprise an agent that has died (e.g., through natural mortality or from a disease). Dead agents are removed from simulations. An immune agent can comprise an agent that is not infected, contagious, and/or susceptible to a disease. Immunity can be acquired by an agent through either surviving a disease cycle (e.g., infected to contagious without transitioning to dead) or vaccination. In many embodiments, a true Boolean value test can be used to generate a count of agents transitioning and/or agents reaching a specific state. The true Boolean value test can also be used to determine positional attributes for counting agents who transition to dead, infected or immune. In many embodiments, an agent can have its Boolean value toggled to false in an initial state and changed to true when in a target state. In many embodiments, a number of time steps that for each state can be determined using a probability. Agents that transition to a death state can be removed from a population dataframe.

When an agent meets conditions for transitioning between states (e.g., it's counter is decremented to zero), it can be labeled as susceptible. Susceptible agents can be compared to an additional condition Boolean array. Agents that are not susceptible can have their disease state counter decremented by one. After an agent transitions out of an immunity state, the agent is labeled as susceptible. This is done by passing an immunity stage to another immunity stage, which switches the Boolean counter and returns the agent into a susceptible state. Double immunizing an agent, while counter intuitive, can avoid issues with finite immunity present in default simulations where immune individuals were also labeled as impossibly susceptible, thereby giving a double count for a susceptible and immune agent. Agents with a probability of death above a predetermined threshold can be classified as a critical case. Critical cases can be placed in an array for reporting.

In many embodiments, a contact-based disease class can be used in an ABM to manage transmission of a contact-based disease between agents. A contact-based disease class can comprise a mathematical probability formula used to spread a disease through agent contact. For example, for any vertex X of a graph, a number of contagious agents (N_{c}) can be counted. Then, each nonimmune agent on the vertex X has a probability of becoming infected, where (contactᵣₐₜₑ) is defined as the contagiousness of the disease. The probability of disease spread can then be defined as:

$$1 - ( 1 - {contact}_{rate} ) * {N}_{c} .$$

For any vertex on a graph, a number of contagious agents can be counted. After counting, one or more susceptible agents can be passed through the mathematical probability formula and receive a probability of catching a contact-based disease. Agents labeled as non-susceptible can be agents that are in an infected state, a contagious state, a natural immunity state, are masked, or are in a quarantine state. In many embodiments, labels for stages immune, quarantined, and masked can be added to a conditional exclusion for susceptible. This is because disease classes does not check for a masked label when identifying agents that can transition from susceptible to infected. A conditional exclusion here specifies that susceptible agents should include agents who are not masked. Masked agents can be defined by a function. The function can determine when an individual is masked using parameters of an agent's float compliance rate and a Boolean value indicating a mandated environment. Processes that generate a contact-based disease class can use NumPy Boolean array comparisons instead of if-loops and for-loops to enhance speed and decrease burdens on a processor. This substitution can reduce burdens on a processor, thereby leading to a faster model and/or a model that has sufficient compute available to incorporates a new variable.

In many embodiments, a custom disease class can be used in an ABM to represent a disease with user-defined attributes. A custom disease class can use a constructor function to collect and initialize disease state values of an agent and can define state values to transition between. A custom disease class can comprise methods that sum up and return the number of agents in a defined state. For example, the methods can comprise pulling lists after each time step for agents in a vertex that are susceptible to disease infection. The method can then compare the list to a number of agents that are contagious in the vertex. These values can then be used to determine a spread of a disease function.

In many embodiments, a custom (e.g., new) disease class can iterate through a list of vertices, and one or more agents on a listed vertex can be given a probability of contamination by the custom disease. The probability can comprise a float probability of contamination between zero and one. A flat rate probability can also be used in a custom disease class. The flat rate can be applied to one or more agents within a predetermined radius instead of (or in addition to) an individual rate for each vertex. In some embodiments, a Boolean condition parameter can be implemented through an if-loop that activates a for-loop, thereby assigning a same given rate to each vertex. In many embodiments, Boolean values and/or Boolean array parameters can be used in a custom disease class for adding conditions and tracking infected agents.

In many embodiments, a composite class can be used in an ABM to simplify disease spread by contact and transitioning agents between states. A composite class can call functions used for finite immunity diseases. The composite class can then pass these functions to additional functions. For example, a first function can contaminate a list of vertices, thereby giving agents on the contaminated vertex a probability of becoming infected. A second function can propagate a disease in a contaminated vertex by passing the function:

$$1 - ( ( 1 - Disease Contageousness ) * ( Number of Contagious Agents on a Vertex ) ) .$$

A third function can move agents at an end of each state to a subsequent state (e.g., from infected to contagious or from immune to susceptible).

In many embodiments, a composite class can be used in an ABM to processes an initialization of intervention variables for vaccination and testing attributes. The composite class can create and/or assign vaccination and testing attributes for agents interacting with a disease having a finite number of time steps in an immunity state. Quarantined agents can be immune for a user-defined number of time steps, and can then transition into a finite disease immunity stage immediately after a quarantine counter is reduced to zero.

In many embodiments, an intervention class can be used in an ABM to implement interventions. In some embodiments, an intervention class can be specialized to implement interventions related to testing and vaccination. The intervention class can implement methods that distribute tests and vaccines to agents within a radius from a vertex. In some embodiments, each vertex can be assigned a testing probability level between zero and one. Agents within that radius that meet a user-defined condition can receive a probability for taking a disease indication test or for receiving a vaccination for the disease. If a probability of taking a disease indication test is below a vertex assigned probability, then the agent is passed to a process that manages test results and responses. The process can check a disease status and user defined compliance rating of an agent. If the agent is currently carrying a disease state and has a compliance rating equal to or above the user defined compliance rating, then the agent enters a quarantine state and receives a positive test status. If the agent is currently carrying a disease state and has a compliance rating below the user defined compliance rating, then the agent receives a positive test status and maintains their current contagious state. In many embodiments, a number of agents that have received a test and/or a number of agents in quarantine are summed and returned. Newly quarantined agents can be stripped of their contagious disease status, thereby reducing their contagious state counters to zero. Quarantined agents can also be transitioned into a natural immunity state after additional time steps.

In embodiments where vaccine interventions are used, user-given conditions can be used to extract qualified agents that exist on the given vertex and assigns them a random uniform probability of taking the vaccine. A compliance value can be defined for each agent as a float from 0.0 to 1.0. Agents who have compliance greater than the compliance value can be selected as being able or willing to take a vaccine intervention. After a probability is assigned, agents are passed to a helper function that can add an array mask for tracking newly vaccinated agents. Agents assigned a probability that is equal to or greater than a user defined probability can be assigned a vaccinated status. A vaccinated status can initialize a vaccine counter and also set a natural immune status to true. A vaccinated status can also remove all contagious disease statuses and then sum a number of vaccinated agents into an array for tracking. An intervention class can also transition and increment newly vaccinated agents after each time step of an ABM. Unlike other agent statuses, vaccine status increments instead of decrements. Once a user defined number of time steps have occurred (e.g., after a time step counter has been incremented a user defined number of times), the intervention class can remove a vaccinated status of an agent These agents can now be labeled as susceptible to being re-infected with the disease.

In many embodiments, a visualization function can be used in an ABM to generate one or more visualizations for monitoring the ABM. A visualization function can take a number of lists as an input. For example, a number of new infections, a number of natural deaths, a total number of deaths, a number of disease-related deaths, a number of critical cases, a number of newly immunized agents, and/or a number of holidays can all be inputs into a visualization function. In many embodiments, inputs can be created and/or updated on a weekly (e.g., a week of time steps) basis. In some embodiments, all or a portion of a year's worth of weekly inputs can be labeled. Holiday weeks can be identified using labels and/or markers. For each input within a range of a weekly infection list (up to 52 weeks), a visualization function can calculate a percentage relative to 52 and can construct weekly labels using an F-string to denote a corresponding year. In many embodiments, visualizations for infections and immunizations can be created. A visualization function can add line traces for the respective metrics to a scatter plot. A visualization function can also add markers for holiday weeks in red. Visualization titles and axis labels can be automatically generated and updated by the visualization function. The visualization function can then construct a figure comprising a bar chart for total tests distributed and line charts for positive tests, negative tests, and/or newly quarantined agents.

A visualization function can also be used to generate a visual representation of testing results over a series of weeks. A number of parameters can be used to generate a visualization of testing results. For example, a total number of distributed tests, a number of positive tests, a number of negative tests, and/or a number of newly quarantined agents can all be used. A visualization function can be used to generate a visual representation of available and distributed tests and/or vaccines. A number of parameters can be used to generate a visualization of distributed test and/or vaccines. For example, a total number of available tests, a total number of available vaccines, a list of distributed tests, and a list of distributed vaccines can all be used. The visualization function can then calculate a total available and distributed quantities for tests and vaccines and generate a bar chart with labels for tests and vaccines. The function can specify positions and widths of the bars. The function can also generate labels for a y-axis, a title, and x-axis tick labels for each bar.

In many embodiments, method 100 can comprise an activity 102 of generating an environment for a for-loop simulation. An environment can be understood as a spatial and geographical context in which agents interact. The environment can act as a backdrop for agent activities by influencing their behavior, movements, and interactions with one another. In many embodiments, census data can be incorporated into an environment. In many embodiments, a default environment for an ABM can be modified to include census data. In order to modify a default environment, one or more shapefiles containing a specified granularity (e.g., state-level, county-level, census tract-level, etc.) of a population distribution can be used to creates a graphable object of population distribution for each shape (e.g., state, county, census tract, etc.). To generate agents that coincide with census population data, a user can define a total graph space of X-Y dimensions (e.g., 100, 500, 1000, etc.). These dimensions can then be passed as arrays of a length of the X-Y dimensions. The larger the X-Y dimension values, the more granular the resulting data.

A user can then create a K-array for a graph space, where K can comprise a number of agents that can be supported in the graph space. In many embodiments, a loop can be generated that iterates through a length of the X and Y dimensions. The loop can also identify a census population for an associated point in the shapefile for each array. To represent a non-rectangular graph-space, a point on the shapefile that has 0 population can have a K-array value of 0, while a shapefile point with a population 10,000 would have a K-array value of 10,000. A final K-array can comprise a 2-dimensional array [X, Y] with each value representing K (e.g., a total census population of that shapefile point).

In order to improve the processing time it takes to generate these graphs for simulation, a K-array (e.g., 2-dimensional X-Y) can be saved as a .csv. A .csv can be used to define the same graph object and rules. For example, a 2-dimensional dataframe can be created from the .csv, and the dataframe read back to create the same graph space with the same K values at each vertex. The K-array can then be converted back into a 1-dimensional array by passing the graph object through a customized function.

A 1-dimensional representation of K values in an environment can then be created (referred to as K_array_flat). Using the 1-dimensional representation, a weighted proportion of population at each vertex can be created. This weighted proportion can define a likelihood of an agent's position being at that vertex during the Agent data frame creation process. This can be done by creating an object that is equal to

$$\frac{K\_array \_flat}{A total sum of K\_array \_flat} .$$

A custom movement function can be implemented to adjust default connections in a graph space. In this way, an agent's likelihood of moving to a vertex can be restricted to only vertices that can hold agents. This movement function can prevent agents from moving to a K=0 space, where the agent would be dropped from the simulation at an end of a time step. The movement function takes in the X-dimension, Y-dimension, the graph object itself, and the K_array_flat object. The function then can iterate through an entire length of K_array_flat and can analyze each connection value in the graph data frame. If the connection value is equal to −1, then the probability of moving that direction is negative, thereby making it an impossible move for an agent. The function can check the 8 possible connections (up, up-right, right, down-right, down, down-left, left, up-left) and weights in the graph object. If the K_array_flat object equals zero at that point, the function can set graph connections and graph weight values at those X-Y dimensions to −1. This, in turn, prevents agents from using this connection as a position movement.

In many embodiments, method 100 can comprise an activity 103 of generating one or more agents. Agents can be generated and saved to a .csv file before a for-loop is initiated to speed up performance of the for-loop, while also starting agents from a same status. This can allow changes made to components such as the disease class, intervention class, or behaviors in the for-loop to be compared to one another because agents and graphs begin from a common starting point. A number of custom classes and functions can be used to accomplish this.

A position generating function can be used to determine what vertex an agent travels to between each timestep. This can be equated to daily travel from home for work, errands, school, or similar movement patterns. The function takes in a graph object, a space for each agent's territory value, and a distance value. The distance value can be user-defined and can also represent an average daily distance traveled by an agent as an integer. For census-based shapefile maps, a real world size of the shape (e.g., an approximate land area in square miles or kilometers) and the X and Y dimensions used to define the graph space can be used to determine a size of each vertex representing a plot of land. For example, if a shapefile represents a 50 mile by 50 mile area and the graph object is 500 by 500 vertices, then each vertex can represent a 10 mile by 10 mile space. An average daily distance traveled by individuals in that real world space can be approximated by using a number of vertices that would equal the length. To continue with the above example, if an average person travels 30 miles per day, then a distance variable would be 3 because 3 vertices equals 30 miles.

The position generating function can first define a position as equal to territory and can then set up a count variable equal to the distance integer. A while-loop can be invoked that creates an array of values that represent connection-values of the 8 neighboring vertices to the defined position value. A position value can be appended to this array for a total array length of 9 representing the 9 available movements an agent can make from each vertex, with the ninth movement being no movement). This array can be filtered to exclude values between −1 and zero, as those values can be considered illegal movements. For example, if an agent is on a vertex on a left edge of a graph, moving further to the left would not be a legal move because it would be equivalent to moving to the rightmost edge of the graph. A count variable can then be decreased by 1, and the while loop continues until that count variable equals zero. After the count variable equals zero, a final position variable is returned by the function. This random walk method can allow for an agent to have a path that is not linear movement in a single direction while at the same time still represent an average distance traveled by a person. A starting value can also be included for each movement, thereby allowing an agent to have a varying distance traveled in each timestep.

An agent reset function can be used to generate an agent data frame that includes agents and their associated features invoked during a for-loop process. An agent reset function can take in a total number of agents being generated and/or simulated, an average distance traveled per time step, and a standard deviation of the average distance. An agent reset function can begin by invoking an agent's object by calling the multi-inheritance class, thereby passing the agent into the multi-inheritance class. Keys for agent age, agent gender, agent territory, agent distance, and/or agent position can then be added to the agent with lists of values that are equal to a total number of agents passed to the agent reset function. Age can be measured in weeks and can comprise a user-defined array or can be a list of ages. Gender can be defined as 0 for males and 1 for females. Territory can comprise a list of values chosen by a random choice function, with possible values equal to a total number of vertices in a graph object space (e.g., an X dimension multiplied by a Y dimension) and can be weighted by a K weighted array. By weighing starting territories to this array, agents can be started in different vertices while at the same time a distribution of starting positions will resemble census data populations. Distance can comprise a list comprising an absolute value of a call to a random choice function, thereby passing an average distance and standard deviation as an integer. Position can comprise a list of values created by applying a position generator function to pass each territory and distance variables through it to derive a total number of agents being generated. After the variables have been defined, agents can be generated and added to an agent object. Agents can then be modified by adding various attributes. For example, a compliance attribute can be added to an agent. A compliance attribute can comprise a user-defined attribute comprising a random and/or uniform distribution of an agent's likelihood of complying with an intervention. As another example, an essential worker attribute can be added to an agent. An essential worker attribute can define an agent as an essential worker who may be exempt from or required to adhere to different interventions. As a further example, a vaccination consistency attribute can be added to an agent. A vaccination consistency attribute can comprise an integer ranging from 0-3. Agents can be given a value of 0 if they do not take an annual flu shot, a value of 1 if they go more than 2 years in between flu shots, a value of 2 if they get semi-annual flu shots, and a value of 3 if they annually receive a flu shot.

In many embodiments, method 100 can comprise an activity 104 of running a for-loop simulation. An ABM can run a simulation for a predefined number of discrete time steps (e.g., days, weeks, hours, months, etc.). In each time step, each agent performs a set of predefined actions according to their attributes, such as moving within the environment, interacting with other agents, or undergoing disease progression (e.g., from susceptible to infected). In each time step, the for-loop can check if agents come into contact with one another, and whether that contact leads to transmission of the disease. The for-loop can apply interventions at each time step.

In many embodiments, method 100 can comprise an activity 105 of generating a visualization. Visualizations as described in activity 101 can be generated after a pre-defined number of time steps have been completed. The visualizations can be displayed in a number of ways. For example, one or more elements of system 200 (FIG. 2) can display the visualizations. As another example, computer system 300 (FIG. 3) can display visualizations.

Turning ahead in the drawings, FIG. 2 illustrates a block diagram of a system 200 that can be employed for disease modeling, as described in greater detail below. System 200 is merely exemplary, and embodiments of the system are not limited to the embodiments presented herein. System 200 can be employed in many different embodiments or examples not specifically depicted or described herein. In some embodiments, certain elements or modules of system 200 can perform various procedures, processes, and/or activities. In these or other embodiments, the procedures, processes, and/or activities can be performed by other suitable elements or modules of system 200.

Generally speaking, system 200 can be implemented with hardware and/or software. Part or all of the hardware and/or software implemented in system 200 can be conventional or part or all of the hardware and/or software can be customized (e.g., optimized) for implementing part or all of the functionality of system 200 described herein.

System 200 can include a web server 201 and one or more web devices 202, 203. Web server 201 and one or more web devices 202, 203 can each be a computer system, such as computer system 300 (FIG. 3), and can each be a single computer, a single server, a cluster or collection of computers or servers, or a cloud of computers or servers. A single computer system can also host each of two or more of web server 201 and one or more web devices 202, 203.

In many embodiments, web devices 202, 203 can be mobile devices. A mobile electronic device can refer to a portable electronic device (e.g., an electronic device easily conveyable by hand by a person of average size) with the capability to present audio and/or visual data (e.g., text, images, videos, music, etc.). For example, a mobile electronic device can comprise at least one of a digital media player, a cellular telephone (e.g., a smartphone), a personal digital assistant, a handheld digital computer device (e.g., a tablet personal computer device), a laptop computer device (e.g., a notebook computer device, a netbook computer device), a wearable user computer device, or another portable computer device with the capability to present audio and/or visual data (e.g., images, videos, music, etc.). Thus, in many examples, a mobile electronic device can comprise a volume and/or weight sufficiently small as to permit the mobile electronic device to be easily conveyable by hand. For example, in some embodiments, a mobile electronic device can occupy a volume of less than or equal to approximately 1790 cubic centimeters, 2434 cubic centimeters, 2876 cubic centimeters, 4056 cubic centimeters, and/or 5752 cubic centimeters. Further, in these embodiments, a mobile electronic device can weigh less than or equal to 15.6 Newtons, 17.8 Newtons, 22.3 Newtons, 31.2 Newtons, and/or 44.5 Newtons. In various embodiments, web devices 202, 203 can comprise a display that is smaller than display device 305 (FIG. 3), thereby facilitating mobility.

Exemplary mobile electronic devices can comprise (i) an iPod®, iPhone®, iTouch®, iPad®, MacBook® or similar product by Apple Inc. of Cupertino, California, United States of America, (ii) a Blackberry® or similar product by Research in Motion (RIM) of Waterloo, Ontario, Canada, (iii) a Lumia® or similar product by the Nokia Corporation of Keilaniemi, Espoo, Finland, and/or (iv) a Galaxy™ or similar product by the Samsung Group of Samsung Town, Seoul, South Korea. Further, in the same or different embodiments, a mobile electronic device can comprise an electronic device configured to implement one or more of (i) the iPhone® operating system by Apple Inc. of Cupertino, California, United States of America, (ii) the Blackberry® operating system by Research In Motion (RIM) of Waterloo, Ontario, Canada, (iii) the Palm® operating system by Palm, Inc. of Sunnyvale, California, United States, (iv) the Android™ operating system developed by the Open Handset Alliance, (v) the Windows Mobile™ operating system by Microsoft Corp. of Redmond, Washington, United States of America, or (vi) the Symbian™ operating system by Nokia Corp. of Keilaniemi, Espoo, Finland.

Further still, the term “wearable user computer device” as used herein can refer to an electronic device with the capability to present audio and/or visual data (e.g., text, images, videos, music, etc.) that is configured to be worn by a user and/or mountable (e.g., fixed) on the user of the wearable user computer device (e.g., sometimes under or over clothing; and/or sometimes integrated with and/or as clothing and/or another accessory, such as, for example, a hat, eyeglasses, a wrist watch, shoes, etc.). A wearable user computer device can comprise a mobile electronic device, and vice versa. However, a wearable user computer device does not necessarily comprise a mobile electronic device, and vice versa.

In specific examples, a wearable user computer device can comprise a head mountable wearable user computer device (e.g., one or more head mountable displays, one or more eyeglasses, one or more contact lenses, one or more retinal displays, etc.) or a limb mountable wearable user computer device (e.g., a smart watch, smart ring, etc.). In these examples, a head mountable wearable user computer device can be mountable in close proximity to one or both eyes of a user of the head mountable wearable user computer device and/or vectored in alignment with a field of view of the user.

In more specific examples, a head mountable wearable user computer device can comprise (i) Google Glass™ product or a similar product by Google Inc. of Menlo Park, California, United States of America; (ii) the Eye Tap™ product, the Laser Eye Tap™ product, or a similar product by ePI Lab of Toronto, Ontario, Canada, and/or (iii) the Raptyr™ product, the STAR 1200™ product, the Vuzix Smart Glasses M100™ product, or a similar product by Vuzix Corporation of Rochester, New York, United States of America. In other specific examples, a head mountable wearable user computer device can comprise the Virtual Retinal Display™ product, or similar product by the University of Washington of Seattle, Washington, United States of America. Meanwhile, in further specific examples, a limb mountable wearable user computer device can comprise the iWatch™ product, or similar product by Apple Inc. of Cupertino, California, United States of America, the Galaxy Gear or similar product of Samsung Group of Samsung Town, Seoul, South Korea, the Moto 360 product or similar product of Motorola of Schaumburg, Illinois, United States of America, and/or the Zip™ product, One™ product, Flex™ product, Charge™ product, Surge™ product, or similar product by Fitbit Inc. of San Francisco, California, United States of America.

Web server 201 and/or web devices 202, 203 can each comprise one or more input devices (e.g., one or more keyboards, one or more keypads, one or more pointing devices such as a computer mouse or computer mice, one or more touchscreen displays, a microphone, etc.), and/or can each comprise one or more display devices (e.g., one or more monitors, one or more touch screen displays, projectors, etc.). In these or other embodiments, one or more of the input device(s) can be similar or identical to input device 303 (FIG. 3). Further, one or more of the display device(s) can be similar or identical to display device 305 (FIG. 3). The input device(s) and the display device(s) can be coupled to the processing module(s) and/or the memory storage module(s) of web server 201 and/or web devices 202, 203 in a wired manner and/or a wireless manner, and the coupling can be direct and/or indirect, as well as locally and/or remotely. As an example of an indirect manner (which may or may not also be a remote manner), a keyboard-video-mouse (KVM) switch can be used to couple the input device(s) and the display device(s) to the processing module(s) and/or the memory storage module(s). In some embodiments, the KVM switch also can be part of web server 201 and/or web devices 202, 203. In a similar manner, the processing module(s) and the memory storage module(s) can be local and/or remote to each other.

Generally speaking, web server 201 can host one or more websites and/or store or run one or more ABMs. For example, web server 201 can host a website that allows users to view one or more visualizations on web devices 202, 203.

Web server 201 and/or web devices 202, 203 can communicate or interface (e.g., interact) with one another through internet 220. Internet 220 can be an intranet that is not open to the public, a mesh network of individual systems, and/or a distributed system. Accordingly, in many embodiments, web server 201 (and/or the software used by such systems) can refer to a back end of system 200 operated by an operator and/or administrator of system 200, and web devices 202, 203 (and/or the software used by such systems) can refer to a front end of system 200. An operator and/or administrator of system 200 can manage system 200, the processing module(s) of system 200, and/or the memory storage module(s) of system 200 using the input device(s) and/or display device(s) of system 200.

Web server 201 and/or web devices 202, 203 can also be configured to communicate with one or more databases. The one or more databases can comprise a product database that contains information about products, items, or SKUs (stock keeping units) sold by a retailer. Data can be deleted from a database when it becomes older than a maximum age, which can be set by an administrator of system 200. Data collected in real-time can be streamed to a database for storage, thereby increasing a storage speed of a database.

The one or more databases can be stored on one or more memory storage modules (e.g., non-transitory memory storage module(s)), which can be similar or identical to the one or more memory storage module(s) (e.g., non-transitory memory storage module(s)) described above with respect to computer system 300 (FIG. 3). Further, the one or more databases can each be stored on a single memory storage module of the memory storage module(s), and/or the non-transitory memory storage module(s) storing the one or more databases or the contents of that particular database can be spread across multiple ones of the memory storage module(s) and/or non-transitory memory storage module(s) storing the one or more databases, depending on the size of the particular database and/or the storage capacity of the memory storage module(s) and/or non-transitory memory storage module(s). In various embodiments, databases can be stored in a cache (e.g., MegaCache) for immediate retrieval on-demand. The one or more databases can each comprise a structured (e.g., indexed) collection of data and can be managed by any suitable database management systems configured to define, create, query, organize, update, and manage database(s). Exemplary database management systems can include MySQL (Structured Query Language) Database, PostgreSQL Database, Microsoft SQL Server Database, Oracle Database, SAP (Systems, Applications, & Products) Database, IBM DB2 Database, and/or NoSQL Database.

Meanwhile, communication between web server 201 and/or web devices 202, 203, and/or the one or more databases can be implemented using any suitable manner of wired and/or wireless communication. Accordingly, system 200 can comprise any software and/or hardware components configured to implement the wired and/or wireless communication. Further, the wired and/or wireless communication can be implemented using any one or any combination of wired and/or wireless communication network topologies (e.g., ring, line, tree, bus, mesh, star, daisy chain, hybrid, etc.) and/or protocols (e.g., personal area network (PAN) protocol(s), local area network (LAN) protocol(s), wide area network (WAN) protocol(s), cellular network protocol(s), powerline network protocol(s), etc.). Exemplary PAN protocol(s) can comprise Bluetooth, Zigbee, Wireless Universal Serial Bus (USB), Z-Wave, etc.; exemplary LAN and/or WAN protocol(s) can comprise Institute of Electrical and Electronic Engineers (IEEE) 802.3 (also known as Ethernet), IEEE 802.11 (also known as Wi-Fi), etc.; and exemplary wireless cellular network protocol(s) can comprise Global System for Mobile Communications (GSM), General Packet Radio Service (GPRS), Code Division Multiple Access (CDMA), Evolution-Data Optimized (EV-DO), Enhanced Data Rates for GSM Evolution (EDGE), Universal Mobile Telecommunications System (UMTS), Digital Enhanced Cordless Telecommunications (DECT), Digital AMPS (IS-136/Time Division Multiple Access (TDMA)), Integrated Digital Enhanced Network (iDEN), Evolved High-Speed Packet Access (HSPA+), Long-Term Evolution (LTE), WiMAX, etc. The specific communication software and/or hardware implemented can depend on the network topologies and/or protocols implemented, and vice versa. In many embodiments, exemplary communication hardware can comprise wired communication hardware including, for example, one or more data buses, such as, for example, universal serial bus(es), one or more networking cables, such as, for example, coaxial cable(s), optical fiber cable(s), and/or twisted pair cable(s), any other suitable data cable, etc. Further exemplary communication hardware can comprise wireless communication hardware including, for example, one or more radio transceivers, one or more infrared transceivers, etc. Additional exemplary communication hardware can comprise one or more networking components (e.g., modulator-demodulator components, gateway components, etc.).

Turning ahead in the drawings, FIG. 3 illustrates a block diagram of a system 300 that can be employed for disease modeling. System 300 is merely exemplary, and embodiments of the system are not limited to the embodiments presented herein. System 300 can be employed in many different embodiments or examples not specifically depicted or described herein. In some embodiments, certain elements or modules of system 300 can perform various procedures, processes, and/or activities. In these or other embodiments, the procedures, processes, and/or activities can be performed by other suitable elements or modules of system 300.

Generally speaking, system 300 can be implemented with hardware and/or software. Part or all of the hardware and/or software implemented in system 300 can be conventional or part or all of the hardware and/or software can be customized (e.g., optimized) for implementing part or all of the functionality of system 300 described herein. When implemented as software, one or more elements of system 300 can be emulated (e.g., reproduced functionally and/or by action via software). For example, a virtual machine having one or more elements described below can be instantiated on one or more elements of system 200 (FIG. 2).

When implemented as hardware, one or more of the elements of system 300 can be coupled together using one or more chassis configured to hold one or more circuit boards and/or serial bus(es). These boards and buses allow the various elements of system 300 to communicate amongst each other to accomplish their intended purposes. While elements of system 300 are described below individually, each can also be integrated into one or more chassis, circuit boards, and/or buses of system 300. On the other hand, one or more elements of system 300 can also be removable (e.g., via a PCI slot on a motherboard and/or a USB port). One or mor elements of system 300 may also be integrated and/or embedded in a different machine or manufacture. Although specific constructions of boards and buses within system 300 are not shown, it should be understood that their construction can be tied to a form factor selected for system 300.

System 300 can take a number of different form factors based on its implementation. For example, system 300 can be implemented as a desktop computer, a laptop computer, a mobile device, and/or a wearable device as described herein. Further, system 300 can comprise a single computer, a single server, a cluster or collection of computers or servers, or a cloud of computers or servers. Typically, a cluster or collection of servers can be used when the demand on 300 exceeds the reasonable capability of a single server or computer, when a distributed structure for system 300 is desired, and/or when parallel computing is desired.

In many embodiments, system 300 can comprise a processor 301, a memory storage 302, an input device 303, a graphics adapter 304, a display device 305, a graphical user interface (GUI) 306, a network adapter 307 and/or audio output 308. Generally speaking, processor 301 can comprise any type of computational circuit. For example, processor 301 can comprise a microprocessor, a microcontroller, a controller, a complex instruction set computing (CISC) microprocessor, a reduced instruction set computing (RISC) microprocessor, a very long instruction word (VLIW) microprocessor, a graphics processor, a digital signal processor, application specific integrated circuits (ASICs), etc. Processor 301 can be configured to implement (e.g., run) computer instructions (e.g., program instructions) stored on memory devices in system 300. At least a portion of the program instructions, stored on these devices, can be suitable for carrying out at least part of the techniques and methods described herein. Architecture and/or design of processor 301 can be compliant with any of a variety of commercially distributed architecture families. For example, a processor can have a 32-bit (x86) architecture and/or a 64-bit (x86-64, IA64, and AMD64) architecture. Processor 301 can be configured to perform parallel computing in combination with other elements of system 300 and/or additional processors. Generally speaking, parallel computing can be seen as a technique where multiple elements of system 300 are used to perform calculations simultaneously. In this way, complex and repetitive tasks (e.g., training a predictive algorithm) can be performed faster and with less processing power than without parallel computing.

Generally speaking, memory storage 302 can comprise non-volatile memory (e.g., read only memory (ROM)) and/or volatile memory (e.g., random access memory (RAM)). The non-volatile memory can be removable and/or non-removable non-volatile memory. Meanwhile, RAM can comprise dynamic RAM (DRAM), static RAM (SRAM), or some other type of RAM.

Further, ROM can include mask-programmed ROM, programmable ROM (PROM), one-time programmable ROM (OTP), erasable programmable read-only memory (EPROM), electrically erasable programmable ROM (EEPROM) (e.g., electrically alterable ROM (EAROM) and/or flash memory), or some other type of ROM. Memory storage 302 can comprise non-transitory memory and/or transitory memory. All or a portion of memory storage 302 can be referred to as memory storage module(s) and/or memory storage device(s). Memory storage 302 can have a number of form factors when used in system 300. For example, memory storage 302 can comprise a magnetic disk hard drive, a solid-state hard drive, a removable USB storage drive, a RAM chip, etc.

Memory storage 302 can be encoded with a wide variety of computer code configured to operate system 300. For example, portions of memory storage 302 can be encoded with a boot code sequence suitable for restoring system 300 to a functional state after a system reset. As another example, portions of memory storage 302 can comprise microcode such as a Basic Input-Output System (BIOS) operable with elements of system 300. Further, portions of the memory storage 302 can comprise an operating system (e.g., a software program that manages the hardware and software resources of a computer and/or a computer network). The BIOS can be configured to initialize and test components of system 300 and load the operating system. Meanwhile, the operating system can perform basic tasks such as, for example, controlling and allocating memory, prioritizing the processing of instructions, controlling input and output devices, facilitating networking, and/or managing files. Exemplary operating systems can comprise software within the Microsoft® Windows®, Mac OS®, Apple® iOS®, Google® Android®, UNIX®, and/or Linux® series of operating systems.

Input device 303 can be configured to allow a user to interact and/or control elements of system 300. A number of devices and be used as input device 303 alone or in combination. For example, input device 303 can comprise a keyboard, a mouse, a touch screen, a microphone, a camera, etc. Input device 303 can be coupled to other elements of system 300 in a number of ways. For example, input device 303 can be coupled via a Universal Serial Bus (USB) port in a wired and/or wireless manner or via a specialized port (e.g., a PS/2 port) depending on the specific device. User inputs through input device 303 can come in a number of forms. For example, when input device 303 comprises a microphone, user input can be received via voice commands and/or a speech to text algorithm. As another example, when input device 303 comprises a camera, user input can be received via bodily movements that are captured and interpreted by system 300.

Generally speaking, graphics adapter 304 can be configured to receive and/or generate one or more elements for display on display device 305. Exemplary embodiments of graphics adapter 304 can comprise devices within the NVIDIA® GeForce® and/or the AMD® RX® series of video cards. In many embodiments, a chipset present on graphics adapter 304 can be configured to perform similar, simultaneous computations in a manner more efficient than other chipsets. For example, rendering a 3D scene on graphics adapter 304 can involve repeated geometric calculations performed in parallel to generate the 3D scene. As another example, repeated mathematical calculations involved in training a predictive algorithm can be performed in parallel on graphics adapter 304 more efficiently than on processor 301. Display device 305 can receive and display signals from graphics adapter 304. A number of devices can be used as display device 305. For example, display device 305 can comprise a computer monitor, a television, a touch screen display, a heads up display (HUD) medium, etc.

In some embodiments, display device 305 can optionally display graphical user interface (GUI) 306. GUI 306 can be a part of and/or displayed by web devices 202, 203 (FIG. 2). With regards to form, GUI 306 can comprise text and/or graphics (image) based user interfaces. For example, GUI 306 can comprise a heads up display (HUD). When GUI 306 comprises a HUD, GUI 306 can be projected onto a medium (e.g., glass, plastic, metal, etc.), displayed in midair as a hologram, and/or displayed on display device 305. GUI 306 can be color, black and white, and/or greyscale. GUI 306 can be implemented as an application running on a computer system, such as computer system 300, web devices 202, 203, and/or web server 201. GUI 306 can also comprise a website accessed through a network (e.g., internet 220). For example, GUI 306 can comprise a visualization website. When GUI 306 allows for modification and/or changes to one or more settings in system 300, it can be referred to as an administrative (e.g., back end) GUI. GUI 306 can also be displayed as or on a virtual reality (VR) and/or augmented reality (AR) system or display. GUI 306 can receive a number of interactions from a user via input device 303. For example, an interaction with a GUI can comprise a click, a look, a selection, a grab, a view, a purchase, a bid, a swipe, a pinch, a reverse pinch, etc.

Network adapter 307 can be configured to connect system 300 to a computer network by wired communication (e.g., a wired network adapter) and/or wireless communication (e.g., a wireless network adapter). Network adapter 307 can be integrated into one or more chassis, circuit boards, and/or buses or be removable (e.g., via a PCI slot on a motherboard). For example, network adapter 307 can be implemented via one or more dedicated communication chips configured to receive various protocols of wired and/or wireless communications. Audio output 308 can be configured to receive and/or generate one or more audio signals for play through a speaker. Exemplary audio outputs 308 can comprise an audio card.

For simplicity and clarity of illustration, the drawing figures illustrate the general manner of construction, and descriptions and details of some features and techniques may be omitted to avoid unnecessarily obscuring the present disclosure. Additionally, elements in the drawing figures are not necessarily drawn to scale. For example, the dimensions of some of the elements in the figures may be exaggerated relative to other elements to help improve understanding of embodiments of the present disclosure. The same reference numerals in different figures denote the same elements.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Furthermore, the terms “include,” and “have,” and any variations thereof, are intended to cover a non-exclusive inclusion, such that a process, method, system, article, device, or apparatus that comprises a list of elements is not necessarily limited to those elements, but may include other elements not expressly listed or inherent to such process, method, system, article, device, or apparatus.

The terms “left,” “right,” “front,” “back,” “top,” “bottom,” “over,” “under,” and the like in the description and in the claims, if any, are used for descriptive purposes and not necessarily for describing permanent relative positions. It is to be understood that the terms so used are interchangeable under appropriate circumstances such that the embodiments of the apparatus, methods, and/or articles of manufacture described herein are, for example, capable of operation in other orientations than those illustrated or otherwise described herein.

The terms “couple,” “coupled,” “couples,” “coupling,” and the like should be broadly understood and refer to connecting two or more elements mechanically and/or otherwise. Two or more electrical elements may be electrically coupled together but not be mechanically or otherwise coupled together. Coupling may be for any length of time, e.g., permanent or semi-permanent or only for an instant. “Electrical coupling” and the like should be broadly understood and include electrical coupling of all types. The absence of the word “removably,” “removable,” and the like near the word “coupled,” and the like does not mean that the coupling, etc. in question is or is not removable.

As defined herein, two or more elements are “integral” if they are comprised of the same piece of material. As defined herein, two or more elements are “non-integral” if each is comprised of a different piece of material.

As defined herein, “real-time” can, in some embodiments, be defined with respect to operations carried out as soon as practically possible upon occurrence of a triggering event. A triggering event can include receipt of data necessary to execute a task or to otherwise process information. Because of delays inherent in transmission and/or in computing speeds, the term “real time” encompasses operations that occur in “near” real time or somewhat delayed from a triggering event. In a number of embodiments, “real time” can mean real time less a time delay for processing (e.g., determining) and/or transmitting data. The particular time delay can vary depending on the type and/or amount of the data, the processing speeds of the hardware, the transmission capability of the communication hardware, the transmission distance, etc. However, in many embodiments, the time delay can be less than approximately one second, two seconds, five seconds, or ten seconds.

As defined herein, “approximately” can, in some embodiments, mean within plus or minus ten percent of the stated value. In other embodiments, “approximately” can mean within plus or minus five percent of the stated value. In further embodiments, “approximately” can mean within plus or minus three percent of the stated value. In yet other embodiments, “approximately” can mean within plus or minus one percent of the stated value.

Although systems and methods for disease modeling have been described with reference to specific embodiments, it will be understood by those skilled in the art that various changes may be made without departing from the spirit or scope of the disclosure. Accordingly, the disclosure of embodiments is intended to be illustrative of the scope of the disclosure and is not intended to be limiting. It is intended that the scope of the disclosure shall be limited only to the extent required by the appended claims. For example, to one of ordinary skill in the art, it will be readily apparent that any element of FIGS. 1-3 may be modified, and that the foregoing discussion of certain of these embodiments does not necessarily represent a complete description of all possible embodiments. For example, one or more of the procedures, processes, or activities of FIG. 1 may include different procedures, processes, and/or activities and be performed by many different modules, in many different orders.

All elements claimed in any particular claim are essential to the embodiment claimed in that particular claim. Consequently, replacement of one or more claimed elements constitutes reconstruction and not repair. Additionally, benefits, other advantages, and solutions to problems have been described with regard to specific embodiments. The benefits, advantages, solutions to problems, and any element or elements that may cause any benefit, advantage, or solution to occur or become more pronounced, however, are not to be construed as critical, required, or essential features or elements of any or all of the claims, unless such benefits, advantages, solutions, or elements are stated in such claim.

Moreover, embodiments and limitations disclosed herein are not dedicated to the public under the doctrine of dedication if the embodiments and/or limitations: (1) are not expressly claimed in the claims; and (2) are or are potentially equivalents of express elements and/or limitations in the claims under the doctrine of equivalents.

What is claimed is:

1. A system comprising:
one or more processors; and
one or more non-transitory memories storing computing instructions configured to communicate with the one or more processors and cause the one or more processors to perform:
defining at least one of one or more classes, one or more objects, or one or more functions;
generating an environment for a for-loop simulation;
generating one or more agents;
running the for-loop simulation using the at least one of the one or more classes, the one or more objects, or the one or more functions; the environment; and the one or more agents; and
generating a visualization of the for-loop simulation.

2. The system of claim 1, wherein the one or more classes comprise age-dependent mortality class.

3. The system of claim 1, wherein the one or more classes comprise a transition class.

4. The system of claim 1, wherein the environment comprises a default environment modified with census data.

5. The system of claim 1, wherein the environment comprises one or more vertices containing the one or more agents.

6. The system of claim 1, wherein the one or more agents comprise an essential worker attribute.

7. The system of claim 1, wherein the one or more agents comprise a vaccination consistency attribute.

8. A method comprising:
defining at least one of one or more classes, one or more objects, or one or more functions;
generating an environment for a for-loop simulation;
generating one or more agents;
running the for-loop simulation using the at least one of the one or more classes, the one or more objects, or the one or more functions; the environment; and the one or more agents; and
generating a visualization of the for-loop simulation.

9. The method of claim 8, wherein the one or more classes comprise age-dependent mortality class.

10. The method of claim 8, wherein the one or more classes comprise a transition class.

11. The method of claim 8, wherein the environment comprises a default environment modified with census data.

12. The method of claim 8, wherein the environment comprises one or more vertices containing the one or more agents.

13. The method of claim 8, wherein the one or more agents comprise an essential worker attribute.

14. The method of claim 8, wherein the one or more agents comprise a vaccination consistency attribute.

15. One or more articles of manufacture including one or more non-transitory, tangible computer readable storage mediums having instructions stored thereon that, in response to execution by one or more processors, cause the one or more processors to perform:
defining at least one of one or more classes, one or more objects, or one or more functions;
generating an environment for a for-loop simulation;
generating one or more agents;
running the for-loop simulation using the at least one of the one or more classes, the one or more objects, or the one or more functions; the environment; and the one or more agents; and
generating a visualization of the for-loop simulation.

16. The one or more articles of manufacture of claim 15, wherein the one or more classes comprise age-dependent mortality class.

17. The one or more articles of manufacture of claim 15, wherein the one or more classes comprise a transition class.

18. The one or more articles of manufacture of claim 15, wherein the environment comprises a default environment modified with census data.

19. The one or more articles of manufacture of claim 15, wherein the environment comprises one or more vertices containing the one or more agents.

20. The one or more articles of manufacture of claim 15, wherein the one or more agents comprise an essential worker attribute.
